(19) United States
(12) Patent Application Publication
(10) Pub. No.: US 2004/0161820 A1
(43) Pub. Date: Aug. 19, 2004

(54) Process for genetic transformation and co-transformation of yeast

(76) Inventors: Gabriela Ileana Sanchez De Rubio, CEP-Sao Paulo (BR); Jose Elisabete Vicente, CEP-Sao Paulo (BR)

Correspondence Address: MORRISON & FOERSTER LLP, 1650 TYSONS BOULEVARD, SUITE 300, MCLEAN, VA, 22102, US

(21) Appl. No.: 10/687,976

(22) Filed: Oct. 20, 2003

(30) Foreign Application Priority Data
Apr. 19, 2001 (BR) PI 0107496-2
Apr. 19, 2002 (WO) WO 02/086057

(51) Int. Cl. C12N 15/74; C12N 1/18

(52) U.S. Cl. 435/69.1; 435/483; 435/254.21

(57) ABSTRACT

The invention is directed to 5S rDNA vectors that can be used to transform yeast strains such as laboratory strains, industrial phototrophic strains, and wild-type strains. 5S rDNA vectors are formed from a 2.1 kb EcoRI-EcoRI S. cerevisiae rDNA fragment that includes the 5S gene and the NTS1 and NTS2 spacers. The p1-9g18 vector has the glycoamylase gene expression cassette of Aspergillus awamory inserted in the HpaI site of the NTS1 spacer. The pA-4 has the geneticin (G418) resistance gene inserted in the HpaI site of the NTS1 spacer, and the pGG7 vector has the geneticin (G418) resistance gene inserted in the HpaI site of the NTS1 spacer, and the glycoamylase gene expression cassette of Aspergillus awmory cloned in the HindIII site of the NTS1 spacer.

### REFERENCE TO RELATED APPLICATIONS

This application claims priority to International Application No. PCT/BR02/00057 filed Apr. 19, 2002, and Brasilian Patent Application No. PI 0107496-2 filed Apr. 19, 2001, both of which are entitled “Process for Genetic Transformation and Co-Transformation of Yeasts,” and both of which are hereby entirely and specifically incorporated by reference.

### BACKGROUND OF THE INVENTION

1 Field of the Invention

This invention is directed to vectors for the expression of nucleic acid sequence in yeast cells and yeast cells transformed by these vectors. The invention is also directed to methods for expression of nucleic acids in yeast with these vectors.

2. Description of the Background

Gene products are prepared in large quantities in microorganisms using a variety of recombinant DNA techniques. Such techniques involve selection of an appropriate host, increasing the number of gene transcripts, improving translation efficiency, and improving the stability of the proteins themselves. To increase the number of gene transcript for high-level production of gene products, it is important to use both an effective transcription promoter and to maximize the number of copies of the gene-expression unit. Typically this comprises increasing the amount of transcription promoter/terminator sequences as well as the gene to be expressed. This increases transcript number as a whole.

For industrial scale production, gene-expression units can be stably maintained in microbial cells. Plasmid vectors are at a disadvantage in this regard and generally stabilized by integration into a chromosome. It has been reported that dozens of copies of a vector could be integrated into the ribosomal RNA gene (rDNA) regions of a yeast cell by using the vector carrying a transformation marker gene in which the promoter region was trancated to reduce expression level (Lopes T. S. et al., Gene, 79, 199-206, 1989; Bergkamp R. J. M. et al., Curr. Genet., 21, 365-370, 1992; Le Dall M. T. et al., Curr. Genet., 26, 38-44, 1994).

However, to achieve high-copy-number integration into the chromosome, integration of the vector into the ribosomal RNA gene region is typically necessary. Otherwise, large numbers of copies will not be obtained when the vector is integrated into other gene loci (Lopes T. S. et al., Gene, 105, 83-90, 1991). Further, introduced genes are generally not sustained due to recombination between their repetitive sequences because integrated vectors exist in a tandem form in the chromosome (Lopes T. S. et al., Yeast, 12, 467-477, 1996). In particular, when microbial cells are cultured under nonselective conditions or microbial growth is slow (for example, when the expression product is present in abundance in the microbial cells), successive cultivation for generations will result in an increase in the ratio of cells without vectors.

Accordingly, when recombinant yeasts are cultured under nonselective conditions (particularly in a large-scale culture), stable maintenance of the integrated vectors is important. An expression unit integrated into the chromosome can be stabilized by shortening the size of vector DNA (Lopes T. S. et al., Yeast, 12, 467-477, 1996). However, further improvements as to high-copy number introduction of the vector into the chromosome and stabilization of the expression units are still to be achieved.

### SUMMARY OF THE INVENTION

The present invention overcomes the problems and disadvantages associated with current strategies and designs and provides new tools and methods for the transformation and expression of genetic sequences in yeast.

One embodiment of the invention is directed to vectors comprising 5S rDNA. These vectors contain a portion of the S. cerevisia geneome, namely, a 2.1 kbp EcoRI-EcoRI fragment containing a 5S rDNA gene and specer regions NTS 1 and NTS 2. Vectors may also contain antibiotic resistance genes such as, but ot limited to, genes for ampicillin resistance, tetracycline resistance, and G418 resistance.

Another embodiment of the invention is directed to methods for the transformation and co-transformation of yeast cells with vectors of the invention. The invention is also directed to yeast cells transformed with vectors of the invention.

Another embodiment of the invention is directed to the expression of genetic sequences of interest from vectors of the invention that have been inserted into yeast cells.

Other embodiments and advantages of the invention are set forth in part in the description, which follows, and in part, may be obvious from this description, or may be learned from the practice of the invention.

### DESCRIPTION OF THE FIGURES

FIG. 1 Construction scheme for 5S rDNA vectors p1-9, p1-9g18, pA-4 and pGG7.

FIG. 2 Construction scheme for 5S rDNA vectors pA-4, p1-9g18, and pGG7.

### DESCRIPTION OF THE INVENTION

As embodied and broadly described herein, the present invention is directed to novel vectors and methods for transforming yeast cells and thereby expressing nucleic acid sequences of interest.

The following description and various examples illustrate the numerous embodiments and variations of the invention, but should not be viewed as limiting the scope of the invention. The invention includes but is not limited to embodiments, modifications and variations understood by one of ordinary skill in the art upon reading and understanding the entirety of the specification, the drawings, the cited references, and the claims.

Vector Construction

5S rDNA vectors of the invention were used to transform various strains of yeast cells including wild-type yeast strains, strains of the genus Saccharomyces, and strains of other non-Saccharomyces genus (e.g. Candida). The basic 5S rDNA vector was formed from a 20.1 kb EcoRI-EcoRI S. cerevisiae rDNA fragments that includes the 5S gene and the NTS1 and NTS2 spacers from S. cerevisiae. In one embodiment, a glycol-amylase gene expression cassette of Aspergillus awamory is inserted into the HpaI site of the NTS1 spacer (p 1-9g18 vector). In another embodiment, the geneticin (G418) resistance gene is inserted into the HpaI site of the NTS1 spacer (pA-4). In another embodiment, the geneticin (G418) resistance gene inserted in the HpaI site of the NTS1 spacer, and the glycol-amylase gene expression cassette of Aspergillus awmory is inserted into the HindIII site of the NTS1 spacer (pGG7).

Bacterial strains used as plasmid hosts included: Escherichia coli DH5: F'lendA1 hsdr17 (rk⁻mk⁻) supE44 thi-1 recA1 gyrA(Nal^{r}) relA1 (lacZYA-argF)U169 deoR (80dlac (lacZ)M15) (Sambrook et al., 1989). Yeast were tested in solid medium YPD and eight industrial strains, from laboratory and seven Amazonian strains were sensitive to 200 μg/ml and 500 μg/ml of G418. Afterwards, the minimum concentration of antibiotic that inhibits the growth (MIC) in liquid medium was determined (Table 1).

TABLE 1

| STRAIN | MIC | |
|---|---|---|
| Saccharomyces cerevisiae | | |
| (laboratory strains) | | |
| YPH252 | ≦50 | μg/ml |
| S288C | 100 | μg/ml |
| Industrial strains | | |
| L105 | 25 | μg/ml |
| L106 | 25 | μg/ml |
| BG 01 | ≦50 | μg/ml |
| SA-01 | ≦50 | μg/ml |
| CR-01 | ≦50 | μg/ml |
| PE-2 | ≦50 | μg/ml |
| Montrachet de la Champagne | ≦50 | μg/ml |
| Fermix | ≦50 | μg/ml |
| Wild-Type Strains (Amazon) | | |
| L77 | 25 | μg/ml |
| L110 | 150 | μg/ml |
| L136 | 25 | μg/ml |
| L151 | 12.5 | μg/ml |
| L69 | 100 | μg/ml |
| L101 | 100 | μg/ml |
| L103 | 150 | μg/ml |

Yeast were chosen to provide continuity to the experiments. Specific strains used included: YPH252, S288C, L105, L106, BG-01, BE-01, CR-01, PE-2, Montrachet de La Champagne, Fermix, L77, L110, L136, L151.

Culture Medium and Conditions

Yeast were grown in liquid medium at 30° C. in a shaker for 16 h, and in solid medium during 48 hours to 72 hours in stove. Culture medium used included: YPD (1% yeast extract, 2% peptone, 2% glucose), YPDA (YPD with 0.5% soluble starch), YPDA-G418 (YPDA with the antibiotic geneticin-G418, 50 μg/ml), and SDA (0.67% base yeast (YNB) without amino acid, glucose 2%, 0.5% soluble starch, with L-histidine, L-adenine, uracil, and L-lysine at 40 μg/ml each, (SDA-THUAL)) (Sherman et al., 1979)). Bacterial cultures were incubated at 37° C. for 16 hours in LB medium (1% tryptone, 1% sodium chloride, 0.5% yeast extract) with 100 μg/ml of ampicillin, or in nutrient agar medium supplemented with 50 μg/ml of kanamycin. To isolate transformant clones, 2 μl/ml of XGAL solution 2%, and 0.5 μl/ml of a solution 0.1 M IPTG were added (Sambrook et al., 1989). Solid medium for yeast or bacterial were identical to liquid mediums but supplemented with 2% bacteriological agar (Sambrook et al., 1989). Cultures were shaken in a New Brunswick Scientific incubator at 100 RPM. All culture media was commercially obtained (Difco Co.) and sterilized at 120° C. for 20 minutes.

Yeast Strain Transformation

Yeast were transformed using the lithium salts method as described in Gietz et al., 1992. Strains that could not be transformed with this methodology were transformed with a modification as described in Gietz et al., 1995. Before plating in selective medium, cells were incubated in one ml of YPD and incubated at 30° C. for 2 h (Sherman et al., 1979). Incubation time was determined as Expression Time (Sherman et al., 1979). Antibiotic concentration used to select transformant yeast were two times the minimum inhibitory concentration (MIC) value, or increased when necessary. Transformants obtained with the vector carrying the glycoamylase expression cassette were confirmed by the presence of an amylose halo around the colony after exposure of plates to iodine vapor.

Co-Transformation of Yeast

Yeast co-transformation was performed as yeast transformations as described in Gietz et al., 1992, and Gietz et al., 1995. Plasmids pAJ50 or YEp13 (to select transformant colonies) were added together with the 5S rDNA vector that carries the heterologous gene. Transformants were select in YPDA-G418, and minimal medium without leucine (SDA-THUA). Expression time was two hours. When selection was performed in minimal medium without leucine, cells were washed with one ml of TE before plating in selective medium. Antibiotic concentrations used were two times MIC values, which was increased when necessary. Co-transformant colonies were identified by the amylose halo around colonies after exposure to iodine vapor.

Bacteria Transformation

In all cloning stages, electroporation was used to transform bacteria by Gene Pulser Transfection Apparatus (BioRad) calibrated to 2.5V, 25 μF and 200 Ω; in 0.2 mm curettes. Cells were prepared according to manufacturer's protocol. Transformants were selected in LB, LB-AP-XGAL-IPTG or NA medium.

DNA Extraction

Purified DNA fragments were preparted using commercially available means (GENE CLEAN; BIO 101, La Jolla, Calif.). Plasmid DNA was isolated and purified by alkaline extraction (Sambrook et Al., 1989). Yeast total DNA was isolated as described by Ausubel et al., 1992.

Enzymes Used in the Process of Yeast Transformation

Restriction and modifying enzymes were supplied by New England Biolabs (Boston, Mass.) or Gibco/BRL (Rockville, Md.). For analysis of mitotic stability of the 5S rDNA, vector transformants were gown in liquid YPD medium, without selective pressure, with shaking for 16 hours. For transformant cells with the geneticin resistance gene (G418^{R}) integrated in the −5 genome, 50-100 μl of 10⁻⁵ culture dilutions were spread in solid YPD medium and YPD-G418 medium. The relation between the number of colonies grown in the selective and non selective medium was calculated. When transformants had the glycoamylase gene inserted, culture dilutions were spread in solid YPDA medium. Percentages of colonies with amylose halos were calculated.

Amylolytic Activity Evaluation

The amylolitic activity of transformant clones was evaluated qualitatively through the presence of an amylose halo in solid medium with 0.5% of starch, after exposure of plates to iodine vapor. Amylolytic activity of transformant clones was evaluated quantitatively through starch consumption in liquid medium. Initial and final cell numbers of yeast cultures were determined spectophotometry at Ab₆₀₀ₙₘ. Cultures were incubated in YPDA medium with shaking for 16 hours. After centrifugation of cell cultures, 100 μl of supernatant was added to 5 ml of I₂ solution (100 L of a solution stock containing 1% I₂, 10% IK in 100 ml of HCl 50 mM prepared immediately before using). Initial and residual starch concentrations were calculated by spectophotometry at Ab₆₆₀ₙₘ, and the quantity of starch consumed by 10⁸ cells was calculated (Chen et al., 1993 and Kim et al., 1988).

Hybridization—Dot Blot and Southern Blot

For dot blot experiments, total yeast DNA was denatured by incubation for 5 minutes at 100° C. and 10 minutes on ice, and blotting on nylon membrane (Hybond N; Amersham Pharmacia Biotech). For Southern Blots, yeast chromosomes were separated in Contour-clamped Homogeneous Electric Fields-CHEF, in Chef Mapper and Chef Mapper XA Pulse Field Electrophoresis Systems (BioRad). Chromosomes samples were prepare according to the manufacturer's protocol. After electrophoresis, DNA was transferred to nylon membranes using a capillary protocol (Sambrook et al., 1989). When vector was introduced to Aspergillus awamory, glycoamylase expression cassette in the yeast chromosome, the probe was 2.1 kb SmaI-BglII fragment of the glycoamylase structural gene. When vector carried only the G418 gene, the probe was the 1.28 kb EcoRI-EcoRI fragment from pUC4K plasmid. Hybridization experiments used AlkPhos Direct (Amersham Pharmacia Biotech).

Determination of the Copy Number of the Vector Integrate in the Genome

To quantify the number of copies of vector integrated into the yeast genome, three different methodologies were compared. In all cases the strains YPH252/CGC and YPH274/CGC with one and two copies of the glycoamylase gene integrated were used as copy number references. Amylose halo diameters were measured as quantification of the starch consume in liquid medium. Densitometric analyses of the intensity signal X-ray films from dot blot using the program One Dscan (Stratagene, La Jolla, Calif.).

Construction of Plasmids and Vectors

Different plasmids were constructed carrying the transformation and co-transformation vectors. These vectors are lineal DNA fragments, with yeast rDNA sequences in their extremities flanking the different heterologous genes. The 5S rDNA vectors are able to introduce multiple copies of the heterologous genes in the rDNA chromosome of the yeast host.

5S rDNA Vector Construction

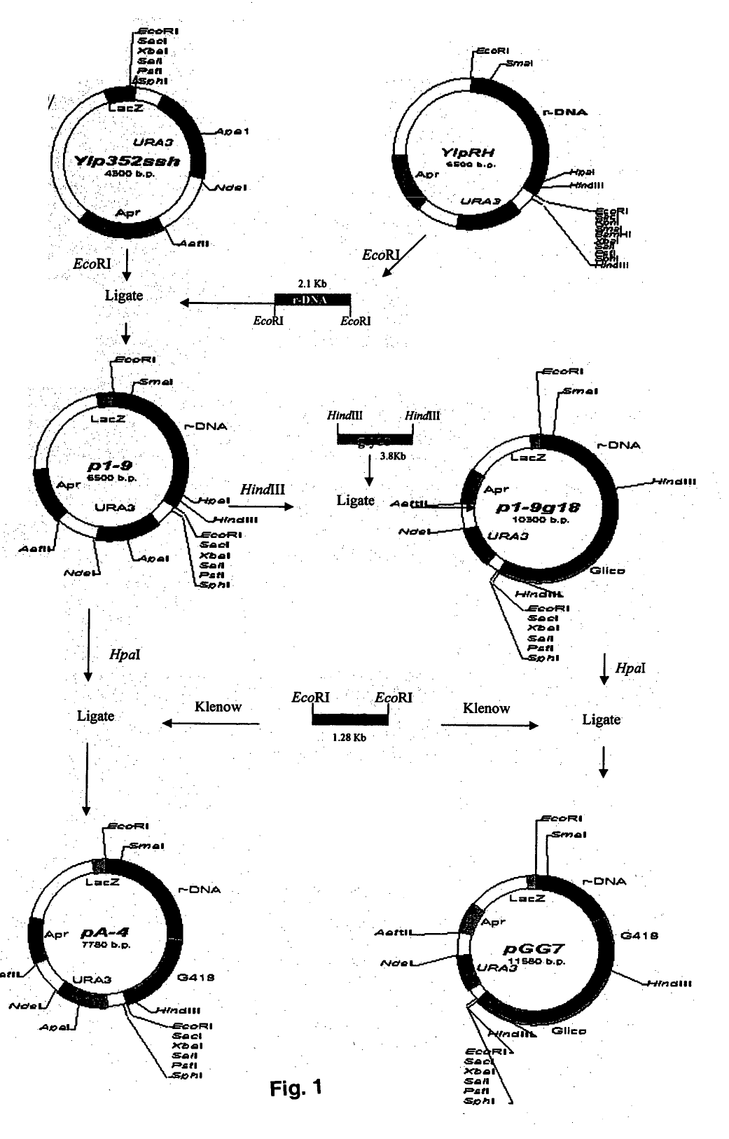

At the beginning several restriction sites were removed. First, the restriction sites HindIII, SmaI, BamHI, and KpnI, present in plasmid YIp352, were removed through digestion with the enzymes HindIII, and KpnI, treatment with the Klenow fragment of DNA polymerase and ligation. The resulting plasmid was referred to as YIp352ssh. Second, the EcoRI site located between structural gene and the terminator sequence of the glycoamylase expression cassette of Aspergillus awamory was removed. This vector was referred to as plasmid YEpG2. To this approach the expression cassette was inserted in the HindIII site of the YEp352SE plasmid and the EcoRI site was inactive as the others sites. This plasmid was referred to as YEp2 EIJ². The next steps were performed as depicted in FIG. 1.

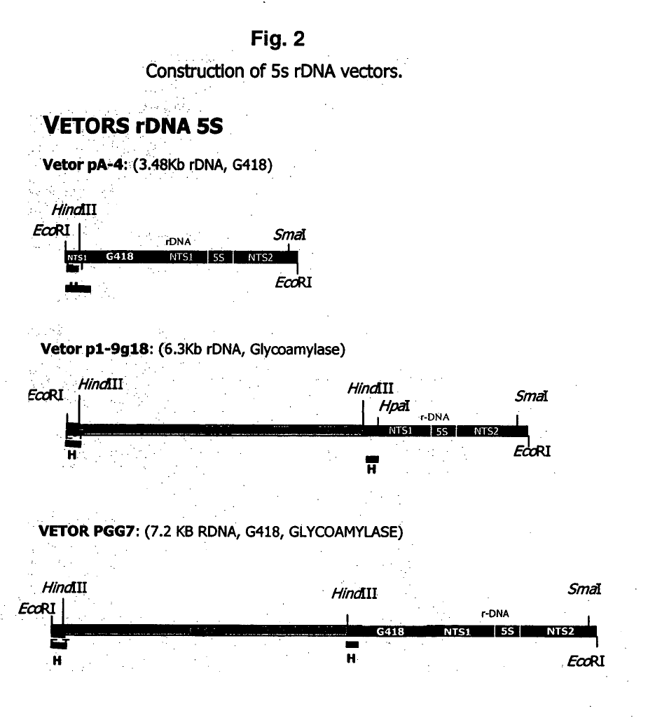

The fragment EcoRI-EcoRI of 2.1 KB from the plasmid YIpRH with the 5S rDNA gene was inserted in the EcoRI site of YIp352ssh plasmid and referred to as p1-9. The glycoamylase expression cassette of A. awamory, present in the fragment HindIII-HindIII of plasmid YEp-EIJ², was inserted into the HindIII site of plasmid p1-9 forming p1-9g18. The 1.28 kb EcoRI-EcoRI fragment that count the gene G418 of plasmid pUC4K, previously treaty with the Klenow fragment of DNA polymerase (fragment EcoRI-EcoRI/Klenow), was inserted in the site HpaI in plasmid p1-9, the new plasmid was pA-4. The fragment EcoRI-EcoRI/Klenow of pUC4K, with the G418 gene, was inserted into the HindIII site of p1-9g18. The resulting plasmid was referred to as pGG7. The 5S rDNA vectors, named p1-9g18, pA-4 and pGG7 (FIG. 2), were obtained after the treatment of each one of the new plasmids with the EcoRI. However, to facilitate the vector DNA preparation, each was subcloned into the EcoRI site of the pUC18 plasmid. Resulting plasmids were referred to as pUC1-9g18, pUCA-4, pUCGG7.

Laboratory strains of S. cerevisiae were transformed with vector 5S rDNA pA-4, pGG7. The transformation study of S. cerevisiae with the new vectors resulted by use of the laboratory lineage YPH252. It was also tested with the prothotrophic standard yeast S. cerevisiae S288C. Selection of transformant clones was performed in complete medium with 120 μg/ml of G418 antibiotic and the transformation efficiency was determined when possible.

In a preliminary analysis the presence of rDNA vectors in the genome of the yeast was confirmed through hybridization Dot Blot using the G418 gene as probe. Mitotic stability of several transformant clones was studied after 100 generations of growth in non selective medium (YPD). Results are shown in Table 2.

TABLE 2

| | YPH252 | | S288C |
|---|---|---|---|
| | Transformation | | Transformation |
| | Efficiency Number | Stability | Efficiency Number of |
| | of transformantes/ | Percent loss | transformantes/μg |
| Vector | μg of DNA | per generation | of DNA |
| pA-4 | 6 × 10² | 0.533 ± 0.02 | 4 × 10² |
| pGG7 | 1.6 × 10³ | 0.023 ± 0.019 | 1 × 10³ |

Integration of the rDNA Vector pA-4 pGG7

Southern blot analysis confirmed that rDNA pA-4, pGG7 vectors integrated into the chromosome XII of the yeast S. cerevisiae YPH252, where several copies of rDNA are located. G418 gene was used as probe.

Transformation of Yeast Strains Isolated from Industrial Processes and from Wild Brazilian Strains with rDNA Vectors pA-4, pGG7

Vector pGG7 showed a larger transformation efficiency with S. cerevisiae. The ability of this vector to transform the previously selected yeast was evaluated. Yeast strains used included those from industrial processes: L1 06, PE2, BE-01, BG-01, CR-01, Montrachet de La Champagne, Fermix and from the Amazonian wild environment: L77, L110, L136, L151. Yeast L77, L110, L136 and L151 belong to Candida, Kluyveromyces, Torulapora and Rhodotorula species. Some yeast were also tested in transformation with other rDNA vectors.

Transformation results and antibiotic concentration used in the selection medium are shown in Table 3.

TABLE 3

Vector

| | G418 | pGG7 | pA-4 |
|---|---|---|---|
| Strain | (μg/ml) | (vector 5S) | (vector 5S) |
| L106 | 75 | + | − |
| Sugar/alcohol | | | |
| PE2 | 100 | + | + |
| Sugar/alcohol | | | |
| SA-01 | 100 | + | + |
| Sugar/alcohol | | | |
| BG-01 | 100 | − | + |
| Sugar/alcohol | | | |
| CR-01 | 100 | − | + |
| Sugar/alcohol | | | |
| Montrachet de | 80 | − | NR |
| la Champagne | | | |
| Vinícola | | | |
| Fermix | 120 | + | NR |
| Panificação | | | |
| L77 Amazon | 150 | + | NR |
| L110 Amazon | 200 | − | NR |
| L136 Amazon | 150 | + | NR |
| L151 Amazon | 180 | − | NR |

+ = presence of transformants
− = absence of transformants

The presence of vector pGG7 in the cell was confirmed through visualization of amylose halos around colonies in solid medium with starch. Transformant clones of yeast L77 and L136 did not have amylose halos. Transformant colonies remained white while negative controls remained blue. In both cases the presence of vector in yeast was confirmed by Dot Blot hybridization.

Mitotic Stability of Vector in Yeast Isolated from Industrial Processes and from Amazonian Wild Environment

Stability of vector pGG7 was verified after 100 generations of growth in non-selective medium (YPD) (Table 4).

TABLE 4

| | Vector pGG7 |
|---|---|
| | (percent loss |
| Strain | per generation) |
| L106 | 0.062 ± 0.015 |
| Sugar/alcohol | |
| PE2 | 0.042 ± 0.001 |
| Sugar/alcohol | |
| SA-01 | 0.181 ± 0.205 |
| Sugar/alcohol | |
| Fermix | 0.056 ± 0.078 |
| Panificação | |
| L77 | 0.012 ± 0.008 |
| Amazon | |
| L136 | 0.790 ± 0.101 |
| Amazon | |

Co-Transformation of S. Cerevisiae with 5S rDNA p1-9g18

Co-transformation of the vector p1-9g18 was developed initially in a S. cerevisiae YPH252 host with selective plasmid pAJ50. pAJ50 has two selection markers, the G418 resistance gene and the auxotrophic marker (LEU⁺). S. cerevisiae S288C transformant cells were isolated in complete medium with 150 μg/ml of G418. In experiments using 3 μg of pAJ50, 3 μg of p1-9g18 vector and 2 hours of expression time, were evaluated. Transformation efficiency was measured as number of transformants per μg of p1-9g18. Co-transformation frequency was measured as number of co-transformants per number of total transformants. Transformation efficiency was measured as number of transformants per μg of pAJ50. Results are shown in the Table 5.

TABLE 5

| STRAIN | MIC | |
|---|---|---|
| Laboratory Strains of | | |
| Saccharomyces cerevisiae | | |
| YPH252 | ≦50 | μg/ml |
| S288C | 100 | μg/ml |
| Strains isolated from | | |
| Industrial Processes | | |
| L105 | 25 | μg/ml |
| L106 | 25 | μg/ml |
| BG 01 | ≦50 | μg/ml |
| SA-01 | ≦50 | μg/ml |
| CR-01 | ≦50 | μg/ml |
| PE-2 | ≦50 | μg/ml |
| Montrachet de la Champagne | ≦50 | μg/ml |
| Fermix | ≦50 | μg/ml |
| Wild-Type Strains | | |
| from the Amazon | | |
| L77 | 25 | μg/ml |
| L110 | 150 | μg/ml |
| L136 | 25 | μg/ml |
| L151 | 12.5 | μg/ml |
| L69 | 100 | μg/ml |
| L101 | 100 | μg/ml |
| L103 | 150 | μg/ml |

Mitotic Stability of Genetic Information Integrated Into the Yeast Genome by Co-Transformation

Stability of vector p1-9g18 in S. cerevisiae YPH252 was determined in 6 transformant clones (YPH252/F1-9g18), after 80 generations of growth without selection pressure (Table 6). For the clones C2 and C12 the loss of plasmid pAJ50 was also determined along the 80 generations (Table 7).

TABLE 6

| | Percent loss/generation of |
|---|---|
| Clones (YPH252/pl-9g18) | vector p1-9g18 |
| C1 | 0.012 ± 0.001 |
| C2 | 0.003 ± 0.001 |
| C4 | 0.007 ± 0.002 |
| C7 | 0.047 ± 0.021 |
| C10 | 0.007 ± 0.001 |
| C12 | 0.042 ± 0.021 |

TABLE 7

| Duplicate Co-Transformation | Percent |
|---|---|
| Frequency of Transformation | 6.1% |
| (Number of co-transformants/ | |
| total transformants) | |
| Efficiency of Transformation | 5.5 × 10² |
| (Number of co-transformantes/ | |
| μg pA-4) | |
| Efficiency of Transformation | 1 × 10⁴ |
| (Number of transformants/μg YEp13) | |

Location of the Integrated Vector

Southern blot analysis confirmed that the not selectable rDNA p1-9g18 vectors integrated into the chromosome XII of the yeast S. cerevisiae YPH252 by co-transformation. The 2.1 kb SmaI-BglII fragment of glycoamylase from the Aspergillus awamory gene was used as probe

Multiple Integration of the p1-9g18 Vector

Densitometric analysis of dot blot hybridization signals to determine the number of copies of the rDNA vector was performed using 0.2 μg and 0.3 μg of DNA total DNA of transformant yeast treated with HindIII and a glycoamylase gene fragment as probe (Table 8).

TABLE 8

| | Number |
|---|---|
| Strain | of Copies |
| YPH252/CGC | 1 |
| YPH274/CGC | 2 |
| P1 | 1 |
| P3 | 2 |
| G1 | 6 |
| G3 | 5 |

Co-Transformation of Industrial Yeast and Yeast Strains from Amazonian Wild Environment

The possibility to introduce heterologous genes in the yeast isolated of the Amazonian biodiversity (L77, L101, L136, L151) and from industrial processes (L106, BG, BE-01, CR-01, PE-2), using the vector p1-9g18 and the selection marker G₄₁₈^{R} of plasmid pAJ50 to select transformants was evaluated. Results of the co-transformations experiments, the antibiotic concentration and the stability of vector are shown in Table 9.

TABLE 9

| | | | Establish- |
|---|---|---|---|
| | | | ment (percent |
| | p1-9g18 Co- | Concentrationof | loss per |
| Strain | Transformants | G418 (μg/ml) | generation) |
| L106 | + | 85 | 0.051 ± 0.014 |
| Sugar/alcohol | | | |
| PE2 | + | 100 | 0.957 ± 1.333 |
| Sugar/alcohol | | | |
| PE2 | + | 100 | 0.957 ± 1.333 |
| Sugar/alcohol | | | |
| SA-01 | − | 100 | − |
| Sugar/alcohol | | | |
| BG-01 | − | 100 | − |
| Sugar/alcohol | | | |
| CR-01 | − | 100 | − |
| Sugar/alcohol | | | |
| Montrachet da | + | 85 | 0.068 ± 0.096 |
| Chardon | | | |
| Vinícola | | | |
| Fermix | + | 100 | 0110 ± 0.091 |
| Panificação | | | |
| L77 | − | 150 | − |
| Amazon | | | |
| L136 | − | 150 | − |
| Amazon | | | |
| L110 | − | 200 | − |
| Amazon | | | |
| L151 | − | 180 | − |
| Amazônica | | | |

+ = presence of transformants
− = absence of transformants

Coupled Co-Transformation

The C2-YPH252/F1-9g18 co-transformant was submitted to a new round of co-transformation. First, co-transformants were grown in non selective medium for 40 generations. LEU⁻ and G418 sensitive clones were isolated indicating the loss of pAJ50. For the second co-transformation, 4 μg of the pA-4 vector as the rDNA integrative non selectable vector and 3 μg of YEp13 (LEU⁺) episomal plasmid were used for auxotrophic selection of transformants. LEU⁺ clones were isolated and tested for G418 resistance in complete solid medium YPD with 120 μg/ml of G418. Antibiotic resistance was confirmed in liquid medium YPD with 120 μg/ml of antibiotic. Several amylolytic LEU⁺G418^{R} clones were obtained confirming the viability of using the 5S rDNA vectors in more than one round of co-transformation. Frequency of integration of the second gene in the same rDNA sequence was about 6% (see Table 7).

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims.

1. A yeast vector comprising a 5S rDNA sequence that contains a fragment of S. cerevisiae rDNA wherein said fragment contains a 5S rDNA gene, an NTS1 spacer NTS1, an NTS2 spacer, and an expression cassette containing a DNA sequence of interest.

2. The vector of claim 1, wherein the NTS1 spacer further incule a glycol-amylase gene expression cassette of Aspergillus awamory.

3. The vector of claim 1, wherein the NTS1 spacer further includes a geneticin (G418) resistance gene.

4. The vector of claim 3, wherein the NTS1 spacer further includes the glycol-amylase gene expression cassette of Aspergillus awmory.

5. The vector of claim 1 which is selected from the group consisting of p1-9, p1-9g18, pA4, pGG, and functional variations, combinations and functional modifications thereof.

6. A yeast cell transformed with the vector of claim 1.

7. A yeast cell transformed with the vector of claim 2.

8. A yeast cell transformed with the vector of claim 3.

9. A yeast cell transformed with the vector of claim 4.

10. A yeast cell transformed with the vector of claim 5.

11. The yeast cell of claim 6 wherein the cell is a strain of yeast selected from the group of strains consisting of laboratory strains, phototrophic strains, industrial strains, wild-type strains, a strain of Saccharomyces genus, and strains of a non Saccharomyces genus.

12. A method for expressing a sequence of interest in a yeast cell comprising:
transforming a yeast cell with a vector of claim 1 to form a transformant; and
expressing the sequence of interest from the transformant.

13. The method of claim 12, wherein the sequence of interest is selected from the group consisting of genes that code for enzymes.

14. The method of claim 12, wherein the yeast cell is selected from the group of yeast cells consisting of wild-type yeast strains, strains of the genus Saccharomyces, and strains of non-Saccharomyces yeast.

15. The method of claim 12, wherein transforming is by homologous recombination in one or multiple copies.

16. The method of claim 12, wherein the transformant is stable for a plurality of generations.

17. The method of claim 16, wherein the plurality is greater than 40.

18. The method of claim 12, wherein the vector is integrated in chromosomal rDNA of said yeast cell.

19. A yeast vector comprising:
a 5S rDNA sequence that contains a fragment of yeast rDNA wherein said fragment contains a 5S rDNA gene and an NTS1 spacer; and
an antibiotic resistance gene.

20. The vector of claim 19, wherein the antibiotic resistance gene is selected from the group consisting of genes that confer resistance to ampicillin, tetracycline, or G418.

21. The vector of claim 19 wherein the fragment is the 2.1 kbp EcoRI-EcoRI fragment of the S. Cerevisiae genome.
